(19) United States
(12) Patent Application Publication
(10) Pub. No.: US 2009/0317457 A1
(43) Pub. Date: Dec. 24, 2009

(54) ANTI-INFLAMMATORY AND WOUND HEALING EFFECTS OF LYMPHOID THYMOSIN B-4

(75) Inventors: Michael Girardi, Madison, CT (US); Robert E. Tigelaar, Madison, CT (US); Michael A. Sherling, Brookline, MA (US); John Shires, Stone Mountain, GA (US); Adrian C. Hayday, Kent (GB); Efstathios Theodoridis, London (GB)

Correspondence Address: WOLF GREENFIELD & SACKS, P.C., 600 ATLANTIC AVENUE, BOSTON, MA, 02210-2206, US

(73) Assignees: Yale University, New Haven, CT (US); King's College London, London (GB)

(21) Appl. No.: 12/471,334

(22) Filed: May 22, 2009

Related U.S. Application Data
(63) Continuation of application No. 10/510,569, filed on Nov. 7, 2005, now abandoned.
(60) Provisional application No. 60/372,614, filed on Apr. 12, 2002.

(51) Int. Cl. A61K 9/127; A61K 38/16; A61K 9/10; A61K 31/7088

(52) U.S. Cl. 424/450; 514/12; 424/484; 514/44 R

(57) ABSTRACT

The invention relates to a method of treating inflammatory conditions in a subject comprising administering to a subject a composition comprising a lymphoid thymosin-β4 polypeptide or a functional lymphoid thymosin-β4 polypeptide variant. The invention also provides a method of promoting wound healing in a subject comprising administering to the subject a composition comprising a lymphoid thymosin-β4 polypeptide or a functional lymphoid thymosin-β4 polypeptide variant. The invention also relates to methods of treating the above mentioned conditions in a subject comprising administering to the subject a nucleic acid encoding a lymphoid thymosin-β4 polypeptide or a functional lymphoid thymosin-β4 polypeptide variant. The invention also relates to pharmaceutical compositions comprising a lymphoid thymosin-β4 polypeptide or a functional lymphoid thymosin-β4 polypeptide variant, or salt thereof, and a pharmaceutically acceptable carrier.

### RELATED APPLICATIONS

This application claims the benefit of the filing date of U.S. Provisional Application No. 60/372,614 filed Apr. 12, 2002 and entitled “Lymphoid Thymosin Beta 4 (LTB4) As An Anti-Inflammatory and Wound Healing Therapy,” by Michael Girardi, Adrian C. Hayday, Michael A. Sherling, John Shires, Efstathios Theodoridis, and Robert E. Tigelaar. The entire teachings of the referenced provisional application are incorporated herein by reference.

### GOVERNMENT SUPPORT

The invention described herein was supported, in whole or in part, by Grant No. KO8AR02072 from the National Institute of Health. The United States Government has certain rights in this invention.

### BACKGROUND OF THE INVENTION

Inflammatory conditions are a significant cause of disabilities that accompany a variety of disease. Inflammatory conditions includes, but are not limited to, a conditions of the ski, lungs, and gut. Inflammatory conditions are currently treated with a diversity of agents including steroids and anti-microbial agents. Even though there are some treatments available for inflammatory conditions, there is still a need for further methods to treat inflammatory conditions.

Wounds are also a significant medical problem. Wounds can result from a diversity of agents and conditions. There is also a need for further methods to promote wound healing.

Throughout this application, various publications are referenced, either by Arabic numerals or directly in the text. Full citations for those publications referenced by Arabic numerals may be found at the end of the specification immediately preceding the claims. The disclosure of all referenced publications is hereby incorporated by reference into this application to describe more fully the art to which this invention pertains.

### SUMMARY OF THE INVENTION

The invention provides a method of treating an inflammatory condition in a subject comprising administering to the subject a therapeutically effective amount of a composition comprising a lymphoid thyinosin-β4 polypeptide, or a functional lymphoid thymosin-β4 polypeptide variant. The inflammatory condition can be any inflammatory condition. In one embodiment, the inflammatory condition is a neutrophil mediated inflammatory condition.

In one embodiment the inflammatory condition is inflammatory bowel disease, such as ulcerative colitis or Crohn's disease. In another embodiment, the inflammatory condition is a joint disease, such as rheumatoid arthritis. In yet another embodiment, the inflammatory condition is a lung disease, such as asthma. In one embodiment, the inflammatory condition is vasculitis.

In another embodiment, the inflammatory condition is a skin condition. In one embodiment, the skin condition is Sweet's syndrome, pyoderma gangrenosum, subcorneal pustular dermatosis, erythema elevatum diutinmm, Behcet's disease or acute generalized exanthematous pustulosis. In another embodiment, the skin condition is a bullous disorder, psoriasis, or a condition resulting in pustular lesions.

In another embodiment, the skin condition is dermatitis, such as contact dermatitis, atopic dermatitis, seborrheic dermatitis, stasis dermatitis or allergic contact dermatitis. In one embodiment, the skin condition is acne.

The invention also provides a method of promoting wound healing in a subject comprising administering to the subject a therapeutically effective amount of a composition comprising a lymphoid thymosin-β4 polypeptide, or a functional lymphoid thymosin-β4 polypeptide variant.

The above described method, of treatment can use any lymphoid thymosin-β4 or a functional lymphoid thymosin-β4 polypeptide variant. In one embodiment, the lymphoid thymosin-β4 polypeptide comprises the amino acid sequence of SEQ ID NO:1 or SEQ ID NO:2. In another embodiment, the lymphoid thymosin-β4 polypeptide is a functional variant of the amino acid sequence of SEQ ID NO:1 or SEQ ID NO:2.

In another embodiment, the lymphoid thymosin-β4 is linked to a protein transduction sequence. In one embodiment, the lymphoid thymosin-β4 polypeptide is linked to a protein transduction sequence via a covalent bond. In another embodiment, the lymphoid thymosin-β4 polypeptide is linked to a protein transduction sequence via a non-covalent bond. In one embodiment, the protein transduction sequence is a heptamer of arginine.

The lymphoid thymosin-β4 polypeptide may be isolated from any suitable source. In yet another embodiment, the lymphoid thymosin-β4 polypeptide is isolated from a lymphoid tissue. In another embodiment, the lymphoid thymosin-β4 polypeptide is isolated from leukocytes or lymphocytes. In yet another embodiment, the lymphoid thymosin-β4 polypeptide is isolated from cells or tissue from the lympho-endoreticular system.

In another embodiment, the lymphoid thymosin-β4 polypeptide is a recombinant polypeptide. In another embodiment, the lymphoid thymosin-β4 polypeptide is a synthetic polypeptide.

In yet another embodiment, the lymphoid thymosin-β4 polypeptide is oxidized.

In one embodiment, the composition used in the above described methods of treatment is administered systemically. In one embodiment, the composition is administered intradermally. In another embodiment, the composition is administered subcutaneously. In another embodiment, the composition is administered orally.

In another embodiment, the composition used in the above described methods of treatment is administered topically. In one embodiment, the composition is in the form of a solution, gel, creme, paste, lotion, spray, suspension, dispersion, salve, hydrogel, a liposome, or ointment formulation. In another embodiment, the lymphoid thymosin-β4 is contained in a liposomal preparation.

In another method, the above described methods of treatment further comprise administering an anti-inflammatory compound that is not lymphoid thymosin-β4 polypeptide.

In one embodiment, the above described methods of treatment further comprise administering a corticosteroid, a retinoid, an antibiotic or a cyclosporine derivative. In one embodiment, the antibiotic is a macrolide derivative.

The invention also comprises a pharmaceutical composition comprising a lymphoid thymosin-β4 polypeptide, or a salt thereof, and a pharmaceutically acceptable carrier.

The invention also comprises a pharmaceutical composition comprising a functional lymphoid thyinosin-β4 polypeptide variant, or a salt thereof, and a pharmaceutically acceptable carrier.

In one embodiment, the pharmaceutical composition further comprises an anti-inflammatory compound that is not lymphoid thymosin-β4 polypeptide.

In one embodiment, the antipharmaceutical composition further comprises a corticosteroid, a retinoid, an antibiotic or a cyclosporine derivative. In one embodiment, the antibiotic is a macrolide derivative.

In one embodiment, the pharmaceutical composition comprises a lymphoid thyinosin-β4 polypeptide linked to a protein transduction sequence. In one embodiment, the lymphoid thymosin-β4 polypeptide is linked to a protein transduction sequence via a covalent bond. In another embodiment, the lymphoid thymosin-β4 polypeptide is linked to a protein transduction sequence via a non-covalent bond. In one embodiment, the protein transduction sequence is a heptamer of arginine.

The invention also comprises the use of a lymphoid thymosin-β4 polypeptide for the manufacture of a medicament for the treatment of an inflammatory condition.

The invention further comprises the use of a lymphoid thymosin-β4 polypeptide for the manufacture of a medicament for the treatment of wounds, e.g., to promote wound healing.

The invention also provides a method of treating an inflammatory condition in a subject comprising administering to the subject a therapeutically effective amount of a nucleic acid encoding a lymphoid thymosin-β4 polypeptide, or a functional lymphoid thymosin-β4 polypeptide variant.

The invention also provides a method of promoting wound healing in a subject comprising administering to the subject a therapeutically effective amount of a nucleic acid encoding a lymphoid thymosin-β4 polypeptide, or a functional lymphoid thymosin-β4 polypeptide variant.

Moreover, the invention provides a method of treating a genetic disorder that causes inflammation of the skin, comprising administering to a subject in need of such treatment a nucleic acid molecule encoding a lymphoid thymosin-β4 polypeptide operably linked to a promoter. In one embodiment, the genetic disorder is selected from the group consisting of Netherton's syndrome, Job's syndrome and Epydermlyosis Bullosa.

### BRIEF DESCRIPTION OF THE DRAWINGS

FIG. 1 shows the differential expression of LTβ4 in activated DETCs. As determined by semi-quantitative RT-PCR, LTβ4 is expressed at higher levels in activated DETCs than in resting DETCs, while the expression of UTβ4 is similar in resting and activated DETCs. Products were sampled at 18, 20, 22, 24, 26 and 28 cycles, establishing a linear range.

FIGS. 2A and 2B show the effects of LTβ4 on λ-Carrageenan induced inflammation. Mice were injected with λ-carrageenan intradermally into the footpad, and assessed for footpad swelling representative of neutrophil mediated inflammation. FIG. 2A shows that a 24 h., mice that received LTβ4 had a significantly reduced footpad swelling than mice receiving either PBS or UTβ4. FIG. 2B shows that at both 6 h. and 24 h., mice that received mLTβ4 had a significantly reduced footpad swelling than mice receiving either PBS or mUTβ4.

FIG. 3 shows the effects of LTβ4 in a TPA (12-O-tetradecanoylphorbol-13-acetate) irritant contact dermatitis assay. ICD was elicited by application of TPA to the ears of naïve mice. FIG. 3 sows that mLTβ4 suppressed TPA-induce ear swelling significantly more than PBS at 6 h. and 24 h., and significantly more than mUTβ4 at 6 h.

FIGS. 4A and 4B show the effect of LTβ4 in a DNFB allergic contact dermatitis assay. ACD (allergic contact dermatitis) was elicited in mice previously sensitized on abdominal skin with DNFB by epicutaneous challenge of the ear skin with low-dose DNFB. FIG. 4A shows that at 6 h., 24 h., and 72 h. after challenge, mice that received LTβ4 demonstrated a significant reduction in ear swelling response when compared to mice receiving PBS or UTβ4. There was no significant suppression of ACD by UTβ4. FIG. 4B shows that mLTβ4 showed a significant reduction in ear swelling response at 24 h. and 72 h. relative to mice that received PBS or mUTβ4.

FIG. 5 shows the effect of LTβ4 on endothelial cell migration using the “scratch wound assay”. The inhibition of mLTβ4 on the migration of human umbilical vein endothelial cells (HUVEC) to close an artificial “wound” in vitro was significantly greater than PBS at 9 h. and 24 h. than mUTβ4 at 24 h.

FIG. 6 shows MALDI-MS analysis of mLTβ4 and mLTβ4.

### DETAILED DESCRIPTION OF THE INVENTION

The invention provides a method of treating a subject (individual) in need of treatment comprising administering to the subject a therapeutically effective amount of a composition comprising a lymphoid thymosin-β4 polypeptide (“LTβ4”). The invention provides a method of treating a subject (individual) in need of treatment comprising administering to the subject a therapeutically effective amount of a nucleic acid encoding a lymphoid thymosin-β4 polypeptide. A subject in need of treatment may be a subject with an inflammatory condition, with a wound or both. The invention also provides a pharmaceutical composition comprising a lymphoid thymosin-β4 polypeptide and a pharmaceutically acceptable carrier. The articles “a,” “an” and “the” are used herein to refer to one or to more than one (i.e., to at least one) of the grammatical object of the article.

As described herein, LTβ4 has been shown to have anti-inflammatory properties and wound healing properties. LTβ4 is generated from the pro-thymosin-beta-4 (pTβ4) gene. The pTβ4 gene also encodes a ubiquitous actin-binding protein (“UTβ4”).

The transcripts for UTβ4 represent >95% of pTβ4 mRNA and is expressed across a spectrum of tissues. LTβ4 has been reportedly limited to lymphoid tissues, such as thymus and spleen, and characterised in two pre B-cell lines [see references 11,12].

UTβ4 is synthesised as a 44 amino acid polypeptide, from which the N-terminal methionine is apparently cleaved. LTβ4 carries an additional 98 base pair exon at the 5′ end of the gene that harbors a start codon 18 base pairs from its 3′ end [reference 13]. Thus, translated LTβ4 is predicted to contain 6 or 7 additional N-terminal amino acids, depending on whether or not the initiator methionine is cleaved from LTβ4, as is the case for UTβ4 [reference 13].

UTβ4 has been shown to inhibit neutrophilic infiltration [reviewed in 7]. Specifically, UTβ4 sulfoxide was identified as the active agent in an immunosuppressive supernatant of glucocorticoid-stimulated monocytes and macrophages, whereupon chemically-synthesized UTβ4 was shown to inhibit neutrophil chemotaxis in vitro, and to inhibit λ-carrageenan-induced edema/inflammation after injection into the mouse footpad [reference 8]. In the latter assay, oxidised UTβ4 was active whereas the native form was not. In addition to its actin-binding capacity and its anti-inflammatory potential, UTβ4 has also been reported to promote the closure of “scratch wounds” in endothelial cell [references 8,9] and keratinocyte monolayers in vitro, as well as full-thickness cutaneous wounds in vivo [reference 10].

Lymphoid Thyinosin-β4 Polypeptide, Functional Variants and Derivatives Thereof

As used herein, the term lymphoid thymosin-β4 polypeptide includes the polypeptide of SEQ ID NO:1 (49 amino acid residues); the polypeptide of SEQ ID NO:2 (49 amino acid polypeptide of SEQ ID NO:1, further comprising a methionine residue at the N-terminus); and functional lymphoid thymosin-β4 polypeptide variants, which are variants of either of these polypeptides sequences (SEQ ID NO:1 or SEQ ID NO:2) and have substantially the same physiological activities, e.g., anti-inflammatory activity and/or wound healing activity, as either the polypeptide of SEQ ID NO:1 or the polypeptide of SEQ ID NO:2.

Functional variants of lymphoid thymosin-β4 polypeptide include polypeptides having amino acid substitutions, deletions, insertions, or inversions with respect to SEQ ID NO:1 or SEQ ID NO:2. For example, functional lymphoid thymosin-β4 polypeptide variants can differ from the amino acid sequence of SEQ ID NO:1 or SEQ ID NO:2 in that at least one amino acid residue of SEQ ID NO:1 or SEQ ID NO:2 is replaced with a conservative amino acid. For example, for the purpose of the present invention, conservative replacements may be made between amino acids, within the following groups:
(I) alanine, serine and threonine;
(II) glutamic acid and aspartic acid;
(III) arginine and lysine;
(IV) asparagine and glutamine;
(V) isoleucine, leucine and valine;
(VI) phenylalanine, tyrosine and tryptophan;
(VII) methionine and other methionine analogues;
(VIII) methionine and other methionine analogues where the sulphur is replaced by Group VIB elements (e.g. Selenium, Tellurium, Polonium);
(IX) oxidised methionine and other oxidised methionine analogues (e.g. Group VIB analogues, methionine sulphoximine);
(X) methionine and other sulphur-containing amino acids (e.g. cysteine) including their oxidisedanalgoues.

Functional lymphoid thymosin-β4 polypeptide variants also include polypeptides that have been modified for the purposes of enhancing therapeutic or prophylactic efficacy, or stability (e.g., ex vivo shelf life and resistance to proteolytic degradation in vivo). Such modified polypeptides, when designed to retain at least one activity of the naturally-occurring form of the protein, are considered functional equivalents of the polypeptide described in more detail herein.

A variety of combinatorial chemistry and combinatorial mutagenesis methods can be used to make polypeptide variants (Wissmamn et al. (1991) Genetics 128: 225-232; Graham et al. (1993) Biochemistry 32: 6250-6258; York et al. (1991) Journal of Biological Chemistry 266: 8495-8500; Reidhaar-Olson et al. (1988) Science 241: 53-57). Such methods, as well as others, can be used to produce a wide variety of lymphoid thymosin-β4 polypeptide variants. Lymphoid thymosin-β4 polypeptide variants can be tested to determine if they are functional variants of the lymphoid thymosin-β4 polypeptide.

For example, whether a change in the sequence of the lymphoid thymosin-β4 polypeptide, or a modification to the lymphoid thymosin-β4 polypeptide, results in a functional variant can be determined by assessing the ability of the variant polypeptide to produce an anti-inflammatory response similar to that produced by lymphoid thymosin-β4 polypeptide.

The lymphoid thymosin-β4 polypeptide may be obtained from any suitable source. In one embodiment, the lymphoid thymosin-β4 polypeptide is isolated from vascular components such as endothelial cells. In another embodiment, the lymphoid thymosin-β4 polypeptide is be isolated from lymphatic vessels or lymphoid tissue. Lymphoid tissue includes, but is not limited to, spleen, lymph nodes, Peyer's patches, tonsils and appendix. In another embodiment, the lymphoid thymosin-β4 polypeptide is isolated from leukocytes or lymphocytes. In one embodiment, the lymphocyte is a B-cell. In another embodiment, the lymphoid thymosin-β4 polypeptide is isolated from cells or tissue from the lympho-endoreticular system. As used herein, the term “isolated” refers to a peptide that is substantially free of cellular material, or culture medium when produced by recombinant DNA techniques.

In one embodiment, the lymphoid thymosin-β4 polypeptide is a recombinant polypeptide. As used herein, the term “recombinant” refers to a polypeptide produced by means of recombinant nucleic acids (e.g., recombinant DNA or RNA methods such as production in a prokaryotic or eukaryotic expression system).

In another embodiment, the lymphoid thymosin-β4 polypeptide is a synthetic polypeptide. Synthetic polypeptides can be chemically synthesized using techniques known in the art, such as Merrifield solid phase f-Moc (9-fluorenylmethoxycarbonyl) or t-Boc chemistry.

In one embodiment, the lymphoid thyinosin-β4 polypeptide is oxidized. The lymphoid thymosin-β4 polypeptide can be oxidized according to any method known to a person having ordinary skill in the art.

The lymphoid thymosin-β4 may be linked to a protein transduction sequence, such as a heptamer of arginine as described in Rothbard et al., “Conjugation of arginine oligomers to cyclosporin A facilitates topical delivery and inhibition of inflammation,” Nature Medicine 69(11):1253-1257 (2000) and Robbins et al., “Peptide Delivery to Tissues via Reversibly Linked Protein Transduction Sequences,” BioTechniques 33:190-194 (2002). The lymphoid thymosin-β4 polypeptide may be linked to a protein transduction sequence via a covalent or non-covalent bond. In one embodiment, the lymphoid thymosin-β4 polypeptide is a fusion protein comprising a protein transduction sequence.

As used herein, the term “protein transduction sequences” refers to, short sequences of amino acids that deliver molecules into cells. See generally, Robbins et al., “Peptide Delivery to Tissues via Reversibly Linked Protein Transduction Sequences,” BioTechniques 33:190-194 (2002), and the references cited therein. A complex comprised of a lymphoid thymosin-β4 polypeptide linked to a protein transduction sequence can be used to introduced the lymphoid thymosin-β4 polypeptide into the cell.

Although some protein transduction based methods rely on fusion of a polypeptide of interest to a sequence which mediates introduction of the protein into a cell, other protein transduction methods do not require covalent linkage of a protein of interest to a transduction sequence. At least two commercially available reagents exist that mediate protein transduction without covalent modification of the protein (Chariot™, produced by Active Motif, www.activemotif.com and Bioporter® Protein Delivery Reagent, produced by Gene Therapy Systems, www.genetherapysystems.com).

Briefly, these protein transduction reagents can be used to deliver proteins, peptides and antibodies to cells including mammalian cells. Delivery of proteins (rather than nucleic acids) to cells has a advantages. For example, many current techniques of gene delivery are based on delivery of a nucleic acid sequence which must be transcribed and/or translated by a cell before expression of the protein is achieved. This results in a time lag between delivery of the nucleic acid and expression of the protein. Delivery of a protein (rather than a nucleic acid encoding a protein) decreases this delay.

Methods of Treatment

The invention provides a method of treating an inflammatory condition, in a subject in need of such treatment (an individual who has an inflammatory condition or a wound), comprising administering to the subject a therapeutically effective amount of a composition comprising a lymphoid thymosin-β4 polypeptide. In one embodiment, the composition is a pharmaceutical composition.

The present invention is useful to treat a wide variety of inflammatory condition, which is any condition resulting from inflammation. An inflammatory condition is any disease state characterized by inflammatory tissues (for example, infiltrates of leukocytes such as lymphocytes, neutrophils, macrophages, eosinophils, masat cells, basophils and dendritic cells) which provoke or contribute to the abnormal clinical and histological characteristics of the disease state. Inflammatory conditions include, but are not limited to, inflammatory conditions of the skin, inflammatory conditions of the lung, inflammatory conditions of the joints, inflammatory conditions of the gut and inflammatory conditions of the eye.

Inflammatory conditions include, but are not limited to, inflammatory arthropathies such as rheumatoid arthritis, psoriatic arthritis, crystal arthritis, reactive arthritis, ankylosing spondylitis, infectious arthritis, juvenile chronic arthritis; connective tissue diseases, such as systemic lupus erythematosis, Sjogren's syndrome, polymyalgia rheumatica, cranial arteritis; vasculitic syndromes, such as Wegener's granulomatosis, polyarteritis nodosa, churg strauss syndrome; respiratory diseases, such as asthma, chronic obstructive pulmonary disease, fibrosing alveolitis, hypersensitivity pneumonitis, sarcoidosis, allergic aspergillosis, cryptogenic pulmonary eosinophilia, bronchiolitis obliterans organising pneumonia; dermatological diseases, such as psoriasis, eczema, urticaria; gastrointestinal diseases, such as ulcerative colitis, Crohn's disease, lupoid hepatitis; haematological disease, such as haemolytic anaemia, idiopathic thrombocytopenic purpura, multiple myeloma, lymphoma/leukaemia; transplantation/prosthetics, such as graft rejection, graft versus host disease, tissue reaction to implanted prostheses; and infections, such as tuberculosis, malaria pneumocystis carinii pneumonia, leprosy.

In one embodiment, the inflammatory condition is a neutrophil mediated inflammatory condition.

In one embodiment, the inflammatory condition is inflammatory bowel disease, such as ulcerative colitis or Crohn's disease.

In one embodiment, the inflammatory condition is a joint disease, such as rheumatoid arthritis.

In one embodiment, the inflammatory condition is a respiratory disease, such as asthma, chronic bronchitis, bronchiolitis, pneumonia, sinusitis, emphysema or any other obstructive airway disease.

In one embodiment, the inflammatory conditions is an inflammatory condition of the eye, including dry eye syndrome.

In one embodiment, the inflammatory condition is a skin condition, such as Sweet's syndrome, pyoderma gangrenosum, subcorneal pustular dermatosis, erythema elevatum diutinum, Behçet's disease or acute generalized exanthematous pustulosis. In another specific embodiment, the skin condition is a bullous disorder, psoriasis, or a condition resulting in pustular lesions.

In another embodiment, the skin condition is dermatitis, such as contact dermatitis, atopic dermatitis, seborrheic dermatitis, stasis dermatitis or allergic contact dermatitis.

In another embodiment, the skin condition is acne, such as acne vulgaris.

In one embodiment, the inflammatory condition is vasculitis, such as hypersensitivity vasculitis or allergic cutaneous vasculitis.

The invention also provides a method of promoting wound healing in a subject comprising administering to the subject a therapeutically effective amount of a composition comprising a lymphoid thymosin-β4 polypeptide. Wounds include, but are not limited to, diabetic wounds, burns, surgical wounds and trauma wounds.

The invention also provides a method of treating septic shock in a subject comprising administering to the subject a therapeutically effective amount of a composition comprising a lymphoid thymosin-β4 polypeptide.

The invention also provides a method of inhibiting or reversing skin aging in a subject comprising administering to the subject a composition comprising a lymphoid thyinosin-β4 polypeptide.

The invention also provides for the use of lymphoid thyinosin-β4 polypeptide for the manufacture of a medicament for the treatment of an inflammatory condition. The inflammatory condition can be any inflammatory condition, including, but not limited to, the inflammatory conditions identified above.

Further, the invention also provides for the use of lymphoid thymosin-β4 polypeptide for the manufacture of a medicament for the treatment of wounds.

A “subject” may be any mammal, including, but not limited to, a human, a cat, a dog, a horse and others. The term “subject” and “individual” are used interchangeably in this application.

As used herein, a “therapeutically effective amount” of lymphoid thymosin-β4 is an amount or dosage sufficient to prevent, ameliorate or eliminate the condition meant to be treated. For example, a therapeutically effective amount of lymphoid thymosin-β4 may be an amount sufficient to prevent, ameliorate or eliminate an inflammatory condition. A therapeutically effective amount of lymphoid thymosin-β4 may also be an amount sufficient to prevent, ameliorate or eliminate a wound. For example, a therapeutically effective amount of lymphoid thymosin-β4 includes an amount sufficient to promote wound healing.

Actual dosage levels of the lymphoid thymosin-β4 polypeptide may be varied so as to obtain an amount which is effective to achieve a desired response in a subject, such as a human. Desired responses may include prevention, amelioration or elimination of an inflammatory condition, or prevention, amelioration or elimination of a wound.

The actual therapeutically effective amount to treat a subject can be determined by one of skill in the art using routine experimentation and may vary by mode of administration. Further, the therapeutically effective amount may vary according to a variety of factors, including the size, age, gender, general health and prior medical history of the individual being treated. Additionally the severity of the condition being treated, as well as the use of other components in an individual's treatment regimen will influence the actual dosage.

A therapeutically effective amount may lie in the range of about 0.001 to about 120 mg/kg bodyweight, e.g., 0.01 to about 120 mg/kg body weight, or in the range of about 0.01 to 50 mg/kg, for example 0.05 to 20 mg/kg. The total daily dose may be given as a single dose, multiple doses, e.g., two to six times per day or by continuous delivery over time (for example, by intravenous infusion or delayed release from a capsule or patch).

The compositions of the present invention can be administered by any route of administration, including, but not limited to, topical administration and systemic administration. The composition may be administered in any unit dosage form, and may be prepared by any of the methods well known in the pharmaceutical art, for example, as described in Remington's Pharmaceutical Sciences (Mack Pub. Co., Easton, Pa., 1990).

In one embodiment, the composition is administered systemically. The term “systemic administration,” or “administered systemically,” as used herein means the administration of a compound such that it enters a subject's bloodstream and, thus, is subject to metabolism and other like processes. Systemic administration includes, for example, intravenous, intraperitoneal, intra-arthrodial (within joints) intramuscular or subcutaneous injections, or inhalation, transdermal or oral administration. In one embodiment, the composition is administered intradermally.

In another embodiment, the composition is administered subcutaneously. In another embodiment, the composition is administered intramuscularly. In another embodiment, the composition is administered orally.

In another embodiment, the composition is administered topically. Examples of formulations used for topical administration include, but are not limited to, a lotion, salve, gel, cream, paste, spray, suspension, dispersion, hydrogel, ointment. In one embodiment, the lymphoid thymosin-β4 polypeptide is contained in a liposomal preparation.

In one embodiment, the above described methods of treatment further comprise administering a compound in addition to the lymphoid thymosin-β4 polypeptide. In one embodiment, the compound is an anti-inflammatory compound such as a corticosteroid, a retinoid, an antibiotic or a cyclosporine derivative. In one embodiment, the antibiotic is a macrolide derivative.

Moreover, the lymphoid thymosin-β4 polypeptide may be administered in conjunction with other compounds which may induce an inflammatory response as a side effect. Compounds which may induce an inflammatory response include, but are not limited to, cytokines such as interferon.

Pharmaceutical Compositions

The invention also provides a pharmaceutical composition comprising a lymphoid thymosin-β4 polypeptide, or a salt thereof, and a pharmaceutically acceptable carrier.

In one embodiment, the pharmaceutical composition further comprises a compound in addition to lymphoid thymosin-β4 polypeptide. In one embodiment the compound is another anti-inflammatory compound, such as a corticosteroid, a retinoid, an antibiotic or a cyclosporine derivative. In one embodiment, the antibiotic is a macrolide derivative.

The phrase “pharmaceutically acceptable” as employed herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation.

Thus, another aspect of the present invention provides pharmaceutically acceptable compositions comprising an effective amount of one or more agents, formulated together with one or more pharmaceutically acceptable carriers (additives) and/or diluents. As described below, the pharmaceutical compositions of the present invention may be specially formulated for administration in solid or liquid form, including those adapted for the following: (1) local administration to the central nervous system, for example, intrathecal, intraventricular, intraspinal, or intracerebrospinal administration (2) oral administration, for example, drenches (aqueous or non-aqueous solutions or suspensions), tablets, boluses, powders, granules, pastes for application to the tongue; (3) parenteral administration, for example, by subcutaneous, intramuscular or intravenous injection as, for example, a sterile solution or suspension; (4) topical application, for example, as a cream, ointment or spray applied to the skin; or (5) opthalamic administration, for example, for administration following injury or damage to the retina. However, in certain embodiments the subject agents may be simply dissolved or suspended in sterile water. In certain embodiments, the pharmaceutical preparation is non-pyrogenic, i.e., does not elevate the body temperature of a patient.

Some examples of the pharmaceutically acceptable carrier materials that may be used include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In certain embodiments, the lymphoid thymosin-β4 polypeptide may contain a basic functional group, such as amino or alkylamino, and are, thus, capable of forming pharmaceutically acceptable salts with pharmaceutically acceptable acids. The term “pharmaceutically acceptable salts” in this respect, refers to the relatively non-toxic, inorganic and organic acid addition salts the lymphoid thymosin-β4 polypeptide. These salts can be prepared in situ during the final isolation and purification of the agents of the invention, or by separately reacting a purified agent of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts and the like. (See, for example, Berge et al. (1977) “Pharmraceutical Salts”, J. Pharm. Sci. 66:1-190.)

The pharmaceutically acceptable salts of the lymphoid thymosin-β4 polypeptide include the conventional nontoxic salts or quaternary ammonium salts of the agents, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the agent which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an agent with the carrier and, optionally, one or more accessory ingredients. In general, the formulations are prepared by uniformly and intimately bringing into association an agent of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a agent of the present invention as an active ingredient. An agent of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration of the agents of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active agents, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Transdermal patches have the added advantage of providing controlled delivery of an agent of the present invention to the body. Such dosage forms can be made by dissolving or dispersing the agents in the proper medium. Absorption enhancers can also be used to increase the flux of the agents across the skin. The rate of such flux can be controlled by either providing a rate controlling membrane or dispersing the agent in a polymer matrix or gel.

Ophthalmic formulations, eye ointments, powders, solutions and the like, are also contemplated as being within the scope of this invention.

Pharmaceutical compositions of this invention suitable for parenteral administration comprise one or more agents of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of an agent, it is desirable to slow the absorption of the agent from subcutaneous or intramuscular injection. This may be accomplished by the use of a liquid suspension of crystalline or amorphous material having poor water solubility. The rate of absorption of the agent then depends upon its rate of dissolution which, in turn, may depend upon crystal size and crystalline form. Alternatively, delayed absorption of a parenterally administered agent form is accomplished by dissolving or suspending the agent in an oil vehicle.

The formulations may also be applied as a topical ointment or cream containing the active ingredient. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream may include, for example, at least 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active ingredient through the skin or other affected areas. Examples of such den-al penetration enhancers include dimethylsulphoxide and related analogues.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as stabilizer. It is also preferred to include both an oil and a fat. Together, the emulsifier(s) with or without stabilizer(s) male up the so-called emulsifying wax, and the wax together with the oil and/or fat make up the so-called emulsifying ointment base which forms the oily dispersed phase of the cream formulations.

Emulgents and emulsion stabilizers suitable for use in the formulation of the present invention include Tween 60, Span 80, cetostearyl alcohol, myristyle alcohol, glycerol mono-stearate and sodium lauryl sulphate.

The choice of suitable oils or fats for the formulation is based on achieving the desired cosmetic properties, since the solubility of the active compound in most oils likely to be used in pharmaceutical emulsion formulations is very low. Thus the cream should preferably be a non-greasy, non-staining and washable product with suitable consistency to avoid leakage from tubs or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Another aspect of the present invention provides a packaged pharmaceutical. In one embodiment, the packaged pharmaceutical comprises (i) a lymphoid thymosin-β4 polypeptide, or functional variant thereof, preferably in therapeutically effective amounts; and (ii) instructions and/or a label for administration of the therapeutic agents for the treatment of subjects having an inflammatory condition.

Methods of Treatment Involving Nucleic Acids

The invention also provides a method of treating an inflammatory condition in a subject comprising administering to the subject a therapeutically effective amount of a nucleic acid encoding a lymphoid thymosin-β4 polypeptide.

In one embodiment, the nucleic acid encoding the lymphoid thyinosin-β4 polypeptide is operatively linked to transcriptional regulatory sequences. In another embodiment, the nucleic acid encoding the lymphoid thymosin-β4 polypeptide is in an expression vector. The inflammatory condition to be treated could be any inflammatory condition, including without limitation, all the inflammatory conditions mentioned in this application.

This invention also provides a method of promoting wound healing in a subject comprising administering to the subject a therapeutically effective amount of a nucleic acid encoding a lymphoid thymosin-β4 polypeptide.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

As used herein, the term “gene” or “recombinant gene” refers to a nucleic acid comprising an open reading frame encoding a polypeptide, including both exon and (optionally) intron sequences. For example, as used herein, the term “gene” includes a nucleic acid encoding a lymphoid thymosin-β4 polypeptide, or a functional variant thereof.

As used herein, the term “vector” refers to a nucleic acid molecule capable of transporting another nucleic acid to which it has been linked. Preferred vectors are those capable of autonomous replication and/or expression of nucleic acids to which they are linked. Vectors capable of directing the expression of genes to which they are operatively linked are referred to herein as “expression vectors”.

A polynucleotide sequence (DNA, RNA) is “operatively linked” to a transcription regulatory sequence when the transcription regulatory sequence controls and regulates the transcription and translation of that polynucleotide sequence. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

“Transcriptional regulatory sequence” is a generic term used throughout the specification to refer to nucleic acid sequences, such as initiation signals, enhancers, and promoters, which induce or control transcription of protein coding sequences with which they are operably linked. In some examples, transcription of a recombinant gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of a protein.

As used herein, the term “tissue-specific promoter” means a nucleic acid sequence that serves as a promoter, i.e., regulates expression of a selected nucleic acid sequence operably linked to the promoter, and which affects expression of the selected nucleic acid sequence in specific cells of a tissue, such as cells of neural origin, e.g. neuronal cells. The term also covers so-called “leaky” promoters, which regulate expression of a selected nucleic acid primarily in one tissue, but cause expression in other tissues as well.

Transcription regulatory sequences are art-recognized and are selected to direct expression of the subject proteins. Accordingly, the term transcriptional regulatory sequence includes promoters, enhancers and other expression control elements. Such regulatory sequences are described in Goeddel; Gene Expression Technology: Methods in Enzymology 185, Academic Press, San Diego, Calif. (1990). For instance, any of a wide variety of expression control sequences may be used in these vectors to express nucleic acid sequences encoding the agents of this invention. Such useful expression control sequences, include, for example, a viral LTR, such as the LTR of the Moloney murine leukemia virus, the LTR of the Herpes Simplex virus-1, the early and late promoters of SV40, adenovirus or cytomegalovirus immediate early promoter, the lac system, the trp system, the TAC or TRC system, T7 promoter whose expression is directed by T7 RNA polymerase, the major operator and promoter regions of phage λ, the control regions for fd coat protein, the promoter for 3-phosphoglycerate kinase or other glycolytic enzymes, the promoters of acid phosphatase, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other proteins encoded by the vector, such as antibiotic markers, should also be considered.

Moreover, the expression vectors can be used to deliver nucleic acids encoding the subject polypeptides. Thus, another aspect of the invention features expression vectors for in vivo or in vitro transfection, viral infection and expression of a subject polypeptide in particular cell types.

Expression vectors may be administered in biologically effective carriers, e.g. any formulation or composition capable of effectively delivering the recombinant gene to cells in vivo or in vitro. Approaches include insertion of the subject gene in viral vectors including recombinant retroviruses, adenovirus, adeno-associated virus, herpes simplex virus-1, lentivirus, mammalian baculovirus or recombinant bacterial or eukaryotic plasmids. Viral vectors transfect cells directly; plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct, electroporation or CaPO₄ precipitation. One of skill in the art can readily select from available vectors and methods of delivery in order to optimize expression in a particular cell type or under particular conditions.

Retrovirus vectors and adeno-associated virus vectors have been frequently used for the transfer of exogenous genes. These vectors provide efficient delivery of genes into cells, and the transferred nucleic acids are stably integrated into the chromosomal DNA of the host. A major prerequisite for the use of retroviruses is to ensure the safety of their use, particularly with regard to the possibility of the spread of wild type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes. Thus, recombinant retrovirus can be constructed in which part of the retroviral coding sequence (gag, pol, env) has been replaced by nucleic acid encoding one of the subject proteins rendering the retrovirus replication defective. The replication defective retrovirus is then packaged into virions through the use of a helper virus by standard techniques which can be used to infect a target cell. Protocols for producing recombinant retroviruses and for infecting cells in vitro or in vivo with such viruses can be found in Current Protocols in Molecular Biology, Ausubel, F. M. et al. (eds.) Greene Publishing Associates, (2000), and other standard laboratory manuals. Examples of suitable retroviruses include pBPSTR1, pLJ, pZIP, pWE and pEM which are known to those skilled in the art. Examples of suitable packaging virus lines for preparing both ecotropic and amphotropic retroviral systems include ψCrip, ψCre, ψ2, ψAm, and PA317.

Furthermore, it has been shown that it is possible to limit the infection spectrum of retroviruses and consequently of retroviral-based vectors, by modifying the viral packaging proteins on the surface of the viral particle (see, for example PCT publications WO93/25234 and WO94/06920). For instance, strategies for the modification of the infection spectrum of retroviral vectors include: coupling antibodies specific for cell surface antigens to the viral env protein; or coupling cell surface receptor ligands to the viral env proteins. Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, can also be used to convert an ecotropic vector into an amphotropic vector.

Moreover, use of retroviral gene delivery can be further enhanced by the use of tissue- or cell-specific transcriptional regulatory sequences which control expression of the gene of the retroviral vector such as tetracycline repression or activation.

Another viral gene delivery system which has been employed utilizes adenovirus-derived vectors. The genome of an adenovirus can be manipulated so that it encodes and expresses a gene product of interest but is inactivated in terms of its ability to replicate in a normal lytic viral life cycle. Suitable adenoviral vectors derived from the adenovirus strain Ad type 5 dl324 or other strains of adenovirus (e.g., Ad2, Ad3, Ad7 etc.) are known to those skilled in the art. Recombinant adenoviruses can be advantageous in certain circumstances in that they can be used to infect a wide variety of cell types, including airway epithelium, endothelial cells, hepatocytes, and muscle cells. Furthermore, the virus particle is relatively stable and amenable to purification and concentration, and as above, can be modified so as to affect the spectrum of infectivity.

Yet another viral vector system is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczlca et al. Curr. Topics in Micro. and Immunol. (1992) 158: 97-129). It is also one of the few viruses that may integrate its DNA into non-dividing cells, and exhibits a high frequency of stable integration.

Another viral delivery system is based on herpes simplex-1 (HSV-1). HSV-1 based vectors may be especially useful in the methods of the present invention because they have been previously shown to infect neuronal cells. Given that many adult neuronal cells are post-mitotic, and thus have been difficult to infect using some other commonly employed viruses, the use of HSV-1 represents a substantial advance and further underscores the potential utility of viral based systems to facilitate gene expression in the nervous system (Agudo et al. (2002) Human Gene Therapy 13: 665-674; Latchman (2001) Neuroscientist 7: 528-537; Goss et al. (2002) Diabetes 51: 2227-2232; Glorioso (2002) Current Opin Drug Discov Devel 5: 289-295; Evans (2002) Clin Infect Dis 35: 597-605; Whitley (2002) Journal of Clinical Invest 110: 145-151; Lilley (2001) Curr Gene Ther 1: 339-359).

The above cited examples of viral vectors are by no means exhaustive. However, they are provided to indicate that one of skill in the art may select from well known viral vectors, and select a suitable vector for expressing a particular protein in a particular cell type.

In addition to viral transfer methods, such as those illustrated above, non-viral methods can be used to express a subject polypeptide. Many nonviral methods of gene transfer rely on normal mechanisms used by cells for the uptake and intracellular transport of macromolecules. Exemplary gene delivery systems of this type include liposomal derived systems, poly-lysine conjugates, and artificial viral envelopes.

It may sometimes be desirable to introduce a nucleic acid directly to a cell, for example a cell in culture or a cell in an animal. Such administration can be done by injection of the nucleic acid (e.g., DNA, RNA) directly at the desired site. Such methods are commonly used in the vaccine field, specifically for administration of “DNA vaccines”, and include condensed DNA (U.S. Pat. No. 6,281,005).

The invention also provides a method of treating a genetic disorder that causes inflammation of the skin, comprising administering to a subject in need of such treatment, a nucleic acid molecule encoding a lymphoid thymosin-β4 polypeptide operably linked to a promoter. In one embodiment, the genetic disorder is Netherton's Syndrome. In another embodiment, the genetic disorder is Job's Syndrome. In another embodiment, the genetic disorder is Epydermlyosis Bullosa.

The invention also provides a method of treating a genetic disorder that causes chronic wounds of the skin, comprising administering to a subject in need of such treatment a nucleic acid molecule encoding a lymphoid thymosin-β4 polypeptide operably linked to a promoter.

### EXAMPLES

The following examples are for illustrative purposes only, and should in no way be construed to be limiting in any respect to the claimed invention.

### Example 1

### Expression of pTβ4 Splice-Variants by Vγ5⁺ DETC

Situated within numerous epithelia of rodents and many other vertebrates are intraepithelial lymphocytes (IELs) composed predominantly of T cells and frequently enriched in those expressing heterodimeric γδ T cell receptors (TCR) [reviewed in 1]. IELs would seem ideally located to maintain epithelial integrity in the face of environmental insults, and it was recently shown that γδ cell deficient mice are highly susceptible to chemically-induced squamous cell carcinomas that can in vitro be directly targeted for cytolysis by cutaneous IELs, specifically Vγ5⁺ dendritic epidermal T cells (DETC) [reference 2].

While DETC can kill dysregulated epithelial cells, they have also been reported to synthesise fibroblast growth factors that may promote epidermal wound healing [reference 3]. Consistent with a role for cutaneous IELs in maintaining epidermal integrity, is the observation that the skin of FVB or NOD mice lacking DETC becomes inflamed and functionally compromised, following of T cell mediated responses to a variety of environmental challenges, including contact allergens and irritants [reference 4]. This potential of DETC to limit internally-induced disruption of epidermal integrity is consistent with earlier observations that DETC can suppress cutaneous infiltration by systemic αβ T cells reactive to auto-antigens expressed in the skin [reference 5]. Nonetheless, the mechanisms of DETC down-regulation of cutaneous inflammation are unknown.

Given the reported biological activities of UTβ4, we hypothesised that activated DETC (dendritic epidermal T cells) might be among the lymphoid cells that express LTβ4, and that such expression might contribute to the anti-inflammatory properties of DETC in vivo. To test this, the expression of UTβ4 and LTβ4 by Vγ5⁺ DETCs freshly isolated from murine skin was analyzed.

### Materials and Methods

DETC line: A single cell suspension of epidermal cells was prepared from normal 3 month-old C57BL/6 mice via trypsin disaggregation and subsequent Histopaque-1083 (Sigma) density gradient centrifugation as previously described [reference 14]. Interface epidermal cells were cultured at 2×10⁵ cells/well in a 24-well plate in 2 ml of complete RPMI media (cRPMI: RPMI 1640 supplemented with 10% fetal bovine serum, 25 mM HEPES, 20 μM L-glutamine, 10 μM sodium pyruvate, 50 μM 2-mercaptoethanol, nonessential amino acids, penicillin/streptomycin) containing 2.0 μg/ml Concanavalin A (Pharmacia) and 10 U/ml murine L-2. The resulting cell line was expanded by serial transfer of half of the well contents to a new well and supplementing each well with 1 ml of cRPMI containing 10 U/ml mIL-2 every 3-4 days. After 10 days, the line was stained with FITC-conjugated MAb F536 (anti-Vγ5; BD PharMingen) and sterile-sorted on a FACS-Vantage™ (Becton Dickinson) using CellQUEST™ software. The sorted Vγ5⁺ cells (99% F536⁺) were expanded for an additional 2 weeks in cRPMI containing 10 U/ml mIL-2. After washing×3 in cRPMI, aliquots of 2.5×10⁶ cells in cRPMI were cultured for 6 h. in either an uncoated well (“resting” cells) or a well previously coated with anti-CD3 MAb 2C11 (BD PharMingen) (“activated” cells).

Preparation of cDNA from DETC. “Resting” and “activated” DETC were harvested and total RNA prepared from each according to the manufacturer's directions using silica gel-based spin columns (RNEasy, Qiagen). cDNA was reverse transcribed from 100 ng of each RNA preparation using oligo-dT (Boehringer) and Omniscript reverse transcriptase (Qiagen) according to the manufacturers' guidelines.

Cycle-Course RT-PCR. For LTβ4, UTβ4 and O-actin, RT-PCR was performed in 10 μl reactions in the presence of 0.25 μM of each of the forward and reverse primers, 250 μM of each of the dNTPs (Abgene, 2.5 MgCl₂, and 0.3 U Taq (Qiagen). Each reaction was supplemented with 0.3331 of ³²P-dCTP (10 mCi/ml, 3000 Ci/mmol, Amersham) and products were amplified for 18, 20, 22, 24, 26 or 28 cycles as follows: 94° C. for 30 s, 61° C. for 30 s, 72° C. for 40 s. Following electrophoresis of the entire reaction on a 4% poly-acrylamide gel, gels were dried on drier (Model 583, BioRad) and the bands visualized by autoradiography on x-ray film (X-Omat AR, Kodal). The primers used were as follows:

| | |
|---|---|
| β-actin forward: | |
| 5″-TCCCTGTATGCCTCTGGTCGTACCAC-3′ | (SEQ ID NO: 3) |
| β-actin reverse: | |
| 5′-CAGGATCTTCATGAGGTAGTCTGTCAG-3′ | (SEQ ID NO: 4) |
| LTβ4 forward: | |
| 5′-TGCCTGTCCAGCGCAGGCACTTG-3′ | (SEQ ID NO: 5) |
| UTβ4 forward: | |
| 5′-CTTCTGAGCAGATCAGACTCTCC-3′ | (SEQ ID NO: 6) |
| Tβ4 (common) reverse: | |
| 5′-CTCTGCTAGCCAGACCATCAGATG-3′ | (SEQ ID NO: 7) |

### Results

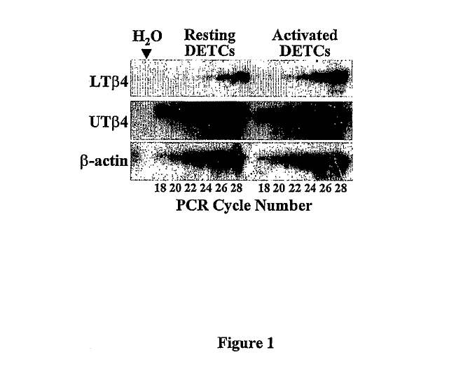

Previous analysis of the gene expression profiles of intestinal TCRαβ⁺ and γδ⁺ IELs (intraepithelial lymphocytes) using SAGE (serial analysis of gene expression) revealed that Tβ4 was expressed at very high levels in both IEL subsets [reference 6]. Since no antibodies directed against T04 are commercially available, we examined the relative levels of Tβ4 splice variants at the mRNA expression level. To determine whether Tβ4 is also expressed by cutaneous γδ⁺ IELs (specifically by Vγ5⁺ DETC that comprise the vast majority of skin IELs in the mouse), and to determine whether TCR-mediated activation of DETC resulted in up-regulated expression of either form of Tβ4, semi-quantitative PCR reactions were carried out on cDNAs prepared from a short-term line of Vγ5⁺ DETC both before (“resting” DETC) and 6 h. after stimulation with anti-CD3 MAb (“activated” DETC). In the method employed, each cDNA is amplified for 18, 20, 22, 24, 26 and 28 cycles in the presence of ³²P-dCTP and primers for either UTβ4, LTβ4, or β-actin; FIG. 1 shows the reaction products visualized by autoradiography, and confirms that across this range, the signal is increasing in a linear fashion relative to cycle number. The data show that both splice variants are expressed by resting and activated DETC. While the overall levels of UTβ4 expression clearly exceed the levels of LTβ4 in both resting and activated cells, UTβ4 expression levels are largely unaffected by TCR-mediated activation. Conversely, LTβ4 expression is clearly up-regulated following activation, with a signal becoming clearly apparent at 22 cycles (with a very faint signal at 20 cycles) that was not apparent prior to activation. The LTβ4 signal from activated DETC is likewise stronger at all cycles thereafter (FIG. 1). The current unavailability of antibodies reproducibly specific for Tβ4 precluded confirmation of these data at the protein level. Nonetheless, the RNA data clearly indicate that DETC express transcripts for both UTβ4 and LTβ4, and that the latter form is induced upon activation via the TCR in vitro.

### Example 2

### Synthesis of Thymosins for Bioassay

Available evidence indicates that the major fraction of UTβ4 undergoes N-terminal methionine processing [references 8,12], but the known rules for aminopeptidase activity [reference 15] do not permit one to male the same assumption for LTβ4. Therefore, in our experiments to determine the biological activities of UTβ4 and LTβ4, we synthesised both N-terminal methionated and un-methionated forms of each polypeptide. The four peptides 104; methionated-UTβ4 (“mUTβ4”), LTβ4; and methionated-LTβ4 (“mLTβ4”)) were synthesized by CS Bio Co. (San Diego, Calif.), using peptide coupling chemistry, and purified (>98%) by reverse phase HPLC (high performance liquid chromatography). The amino acid sequences were as follows: UTβ4: N-SDKPDMAEIEKFDKSKLKKTETQEKNPL-PSKETIEQEKQAGES-C (SEQ ID NO:1); mUTβ4: N-MSDKPDMAEIEKFDKSKLKKTETQEINPL-PSIETIEQEKQAGES-C (SEQ ID NO:2); LTβ4: N-LLPATMSDKPDMAEIEKFDKSKLKKTE-TQEKNPLPSKETIEQEKQAGES-C (SEQ ID NO:8); and mLTβ4: N-MLLPATMSDKPDMAEIEIFDKSKILITE-TQEKNPLPSKETIEQEKQAGES-C (SEQ ID NO:9). In each case, the non-methionated forms lacked the N-terminal residue shown in parentheses. The peptides, were verified by matrix assisted laser desorption ionization electro-spray (MALDI-ES) in W. M Keck Foundation Biotechnology Resource Laboratory at Yale. The average molecular weights and mass errors as determined by matrix assisted laser desorption ionization electro-spray (MALDI-ES) of the synthesized Tβ4 peptides were as follows: UTβ4: 4962.4 Daltons, 0.009%; mUTβ4: 5093.13 Daltons, 0.032%; LTβ4: 5592.52 Daltons, 0.026%; mLTβ4: 5722.92 Daltons, 0.012%. The purities of UTβ4, LTβ4, mLTβ4 were: 90%, 99.4%, 98.8% and 98.5% respectively.

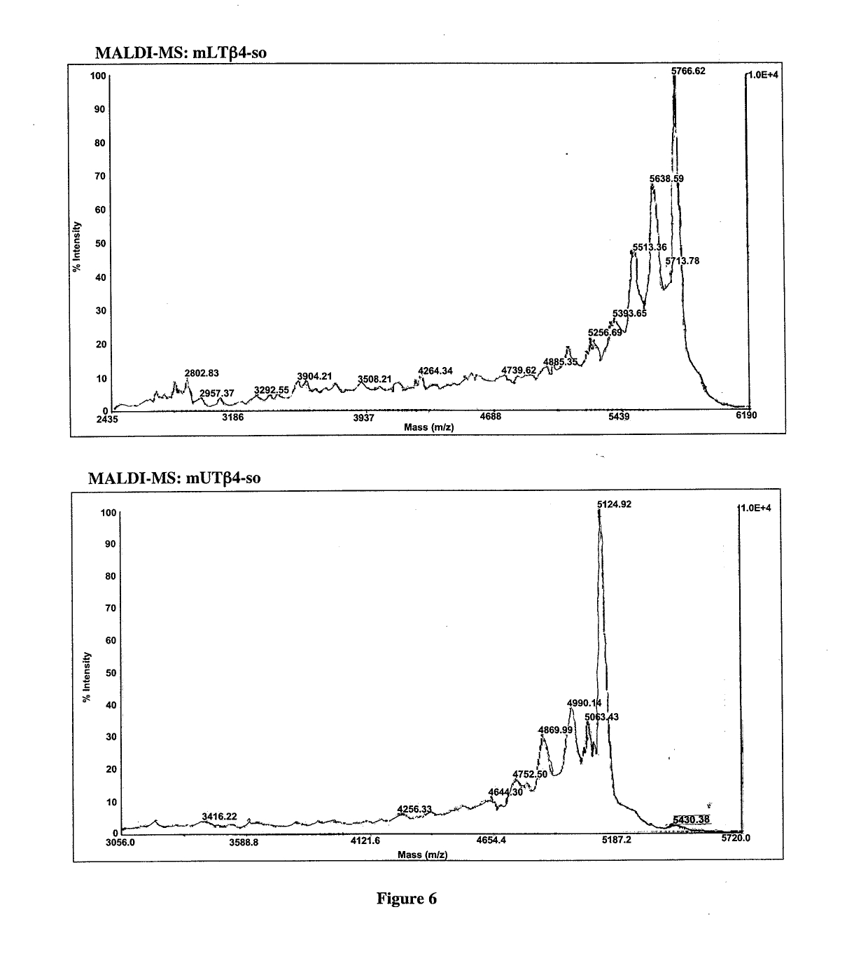

Because of previous findings demonstrating the anti-inflammatory effects of oxidised UTβ4, all peptides were oxidised prior to use, by the addition of an equal volume of 30% H₂O₂ and distilled H₂O for 5 min at room temperature, dried under vacuum centrifugation [as described in 8], and analyzed by MALDI-MS (matrix assisted laser desorption ionization mass spectrometry). The results of the MALDI_MS analysis are shown in FIG. 6.

Oxidation of Thymosins

Once a methionine residue is exposed to 30% hydrogen peroxide, it undergoes an oxidation reaction to form methionine sulfoxide. In this reaction, methionine gains one additional oxygen moeity, which adds 16 atomic mass units (a.m.u's) to its molecular weight.

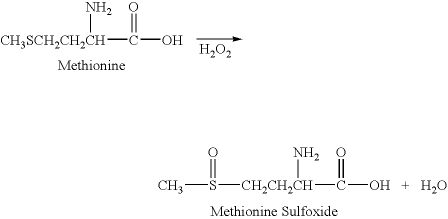

If the oxidation reaction is carried too long, methionine sulfoxide can oxidize to methionine sulfone. In this reaction, methionine sulfoxide gains one additional oxygen moeity, which adds 16 atomic mass units (a.m.u's) to its molecular weight.

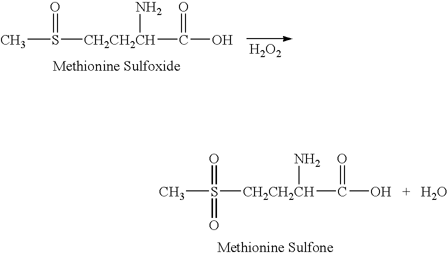

When peptides that contain multiple methionines, are treated with 30% hydrogen peroxide, all methionine residues will oxidize to methionine sulfoxide before any one

methionine sulfoxide oxidizes to methionine sulfone. This hypothesis is derived from Gibb's free energy theory, which states that the energy necessary to oxidize a methionine to methionine sulfone is much higher than the energy it takes to oxidize a methionine to methionine sulfoxide.

Thymosins have no oxidisable amino acids (e.g. cysteine residues) other than methionine. To assess whether the extent of oxidation is consistent de facto with oxidation only of methionines, the change in molecular weight in each polypeptides after oxidation was determined. As seen in Table 1, the change in molecular weight in each case is consistent with oxidation of each of its methionine residues to methionine sulfoxide.

TABLE 1
Oxidations of Thymosin Peptides

| | Met | Original | | | # of |
|---|---|---|---|---|---|
| | residues | MW* | Oxidized MW | Difference | Oxidations† |
| UTβ4 | 1 | 4962.40 | 4980.48 | 18.08 | 1.13 |
| mUTβ4 | 2 | 5093.13 | 5124.92 | 31.79 | 1.99 |
| LTβ4 | 2 | 5592.52 | 5621.96 | 29.44 | 1.84 |
| mLTβ4 | 3 | 5717.68 | 5766.62 | 48.94 | 3.06 |

*MW = molecular weight
†calculated as the difference in MW divided by the MW of oxygen (15.9994)

### Example 3

### Activities of UTβ4 and LTβ4 in λ-Carrageenan-Induced Inflammation

Prior to this invention, no one had evaluated the effects of LTβ4 on neutrophil mediated inflammation. Neutrophils mediate many inflammatory reactions found in skin diseases, including neutrophilic dermatoses, pustular eruptions in systemic diseases, and neutrophilic drug eruptions. In his 2000 review article of neutrophilic dermatoses (ND), Wallach presents a classification of neutrophil mediated dermatoses including Sweet's Syndrome, Pyoderma Gangrenosum, Subcorneal Pustular Dermatosis and Erytherna Elevatum Diutinum (Wallach, D (2000) Neutrophilic Dermatoses: An Overview. Clinics in Dermatology 18:229-231). In Callen's review of “Pustual Eruptions in Systemic Disease,” neutrophils were identified in playing a role in skin manifestations of Ulcerative Colitis, Rheumatoid Arthritis, and Bechet's Disease (Callen, (2000) Pustular Eruptions in Systemic Disease. Clinics in Dermatology 18:349-353). Finally, in Roujeau's review of “Neutrophilic Drug Eruptions,” neutrophilic eruptions were identified diseases such as Drug-Induced Sweet's Syndrome, Drug-Induced Pyoderma Gangrenosum, as Acute Generalized Exanthematous Pustolosis. (See Roujeau, J C (2000) Neutrophilic Drug Eruptions. Clinics in Dermatology 18:331-337 and Roujeau, J C (2000) Polymorphonuclears: Structure, Function, and Mechanisms of Involvement in Skin Diseases. Clinics in Dermatology 18:233-244.) Skin diseases with neutrophilic inflammation can also be classified according to the origin of pathogenesis such as infectious, vasculitic, auto-antibody and complement mediated, T-cell mediated neutrophilic activation, and mast cell release.

Since neutrophils mediate so many dermatological disorders, newer therapies that reduce neutrophil mediated inflammation could target vasculitis, bullous disorders, and pustular psoriasis without the toxic systemic effects of corticosteroids. In order to test the ability of the lymphoid and ubiquitous isoforms of Tβ4 to reduce neutrophil mediated inflammation. a murine model of neutrophil mediated inflammation was employed.

### Materials and Methods

Experimental groups of 8-10 female BALB/c mice, 8-10 weeks of age, were housed in temperature controlled rooms and given food and water ad libitum. Observations and measurements were made by an investigator blinded to the experimental group.

λ-Carrageenan injection. After measuring the baseline thickness of the hind footpads with a spring-loaded engineer's micrometer, mice were injected subcutaneously in both hind paws with 340 μg of λ-carrageenan in 40 μl. 6 h., 24 h., and 48 h. after the injection of λ-carrageenan, footpads were re-measured and swelling calculated by subtracting the baseline from the experimental measurements. For each mouse, increases in right and left footpad thickness were averaged. Thymosin peptides were injected as previously described [reference 8]: (i) intraperitoneally with 100 μl of 3.50×10⁻⁵ M peptide solution, 30 minutes before footpad injection; (ii) intradermally into the footpad with 40 μl of 8.75×10⁻⁵ M peptide solution and λ-carrageenan at time 0, and (iii) intraperitoneally with 100 μl of 3.50×10⁻⁵ M peptide solution, 6 h. after footpad injection. For each group, the mean increase in footpad thickness was calculated, and compared to other groups using a one-tailed Student's t-test. The experiment was repeated four times.

Statistics. Groups were compared for differences in the meaning using a one-tailed Student's t test; p<0.05 was considered statistically significant.

### Results

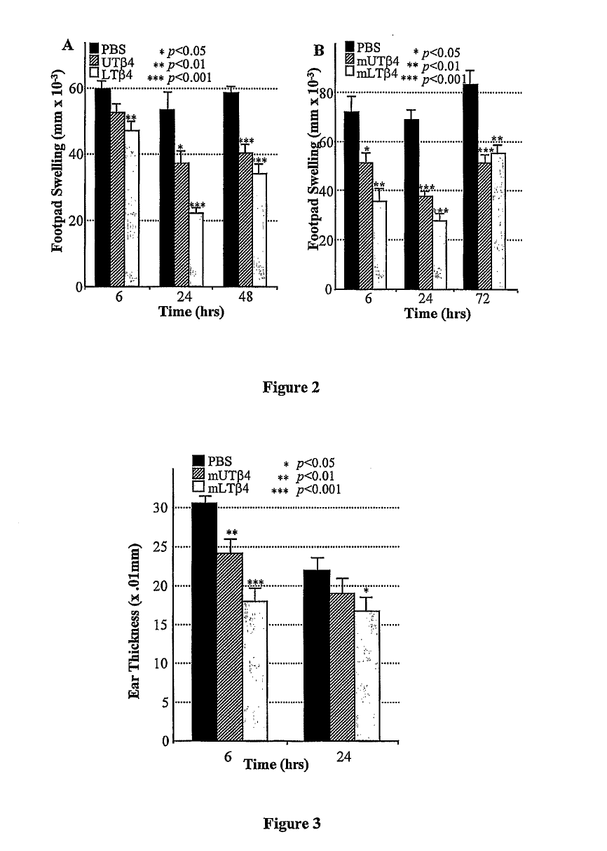

Oxidised UTβ4 and LTβ4 were compared to PBS vehicle alone for their capacity to suppress inflammation induced by intradermal injection with λ-carrageenan into the footpad. λ-carrageenan injection results in an edematous, neutrophil-rich inflammatory response that can be quantitated as footpad swelling over baseline. As seen in FIG. 2A, this study confirmed the reported anti-inflammatory activity of UTβ4 [reference 8]; at 6 h., 24 h., and, 48 h., UTβ4 suppressed footpad inflammation by 12% (p=0.048), 30% (p=0.012), and 31% (p<0.0001), respectively, relative to PBS. Additionally, this study demonstrated that LTβ4 also has substantial anti-inflammatory activity. Suppression relative to PBS at 6 h., 24 h., and, 48 h., respectively, was 21% (p=0.0053), 58% (p<0.0001), and 42% (p<0.00001) (FIG. 2). Relative to UTβ4, LTβ4 was more effective at suppressing inflammation at every time point, with the difference in footpad thickness at 24 h. being statistically significant (0.22±0.03 vs. 0.37±0.03, p=0.0002).

FIG. 2B shows that methionated UTβ4 also has anti-inflammatory activity; footpad thickness was suppressed by 29% (p=0.011), 45% (p<0.00001), and 38% (p=0.00053) at 6 h., 24 h., and, 48 h., respectively. Likewise, mLTβ4 was anti-inflammatory as footpad thickness was suppressed by 50% (p=0.0011), 59% (p<0.0001), and 34% (p=0.0053) at 6 h., 24 h., and, 48 h. At the 6 h. and 24 h. time points, the anti-inflammatory activities of mLTβ4 were significantly greater than those of mUTβ4 (p=0.035 and 0.017, respectively).

The results of this experiment are also summarized in Table 2.

TABLE 2
λ-Carrageenan Footpad Thickness and P-Values

| Un-Methionated Thymosin Experiment (A) | | | | | Methionated Thymosin Experiment (B) | | | |
|---|---|---|---|---|---|---|---|---|
| | Thickness | | P values | | Thickness | | P values | |
| Time | (nm +/− SE) | | vs. PBS | vs. uTβ4-so | (mm +/− SE) | | vs. PBS | vs. mTβ4-so |
| Six | PBS | 0.60 ± 0.02 | * | * | PBS | 0.72 ± 0.06 | * | * |
| Hours | uTβ4- | 0.53 ± 0.03 | 4.81 × 10⁻² | * | mTβ4-so | 0.51 ± 0.05 | 1.11 × 10⁻² | * |
| | so | | | | | | | |
| | LTβ4- | 0.47 ± 0.04 | 5.21 × 10⁻³ | NS | mTβ4-so | 0.36 ± 0.06 | 1.07 × 10⁻³ | 1.47 × 10⁻² |
| | so | | | | | | | |
| Twenty- | PBS | 0.53 ± 0.03 | * | * | PBS | 0.69 ± 0.03 | * | * |
| Four | uTβ4- | 0.37 ± 0.03 | 1.19 × 10⁻² | * | mTβ4-so | 0.38 ± 0.01 | 8.95 × 10⁻⁶ | * |
| Hours | so | | | | | | | |
| | LTβ4- | 0.22 ± 0.03 | 6.03 × 10⁻³ | 2.06 × 10⁻⁴ | mTβ4-so | 0.28 ± 0.04 | 4.24 × 10⁻⁵ | 1.70 × 10⁻² |
| | so | | | | | | | |
| Forty- | PBS | 0.58 ± 0.05 | * | * | PBS | 0.83 ± 0.06 | * | * |
| Eight | uTβ4- | 0.41 ± 0.04 | 6.01 × 10⁻³ | * | mTβ4-so | 0.51 ± 0.02 | 5.34 × 10⁻⁴ | * |
| Hours | so | | | | | | | |
| | LTβ4- | 0.34 ± 0.04 | 2.03 × 10⁻⁶ | NS | mTβ4-so | 0.55 ± 0.03 | 5.32 × 10⁻³ | NS |
| | so | | | | | | | |

NS = not significant

### Example 4

### Activities of UTβ4 and LTβ4 in Irritant Contact Dermatitis (ICD)

Prior to this invention, no one had evaluated the role of LTβ4 and UTβ4 in the abatement of irritant contact dermatitis, although irritant contact dermatoses are important to study, since they represent 20-70% of all occupational diseases (Corsini et al. (1998) Cytokines and irritant contact dermatitis. Toxicology Letters 102-103:277-282). Irritant contact dermatitis can result from occupational exposures to plant materials, plastics, acrylics, water-based paints, metal working fluids, and synthetic work clothes. (Koh, D., Gob, C. (1998) Occupational Dermatology. Clinics in Dermatology 16:113-118). The inflammatory mechanism of irritant dermatitis is poorly understood, although some researchers postulate that environmental irritants induce epidermal keratinocytes to release cytokines. These cytokines are inflammatory (TL-1 TNF-a), chemotactic (IL-8, IP-10), growth promoting (IIr6, IL-7, IL-15, GM-CSF and TGF-a), and regulatory of humoral and cell mediated immune responses (Corsini (1998), cited supra). These cytokines promote cellular infiltrates which can result in epidermal damage, thickening and scaling of the skin, and/or vasodilatation, which can results in erythema and edema of the skin.

### Materials and Methods

Experimental groups of 8-10 female BALB/c and FVB mice, 8-10 weeks of age, were housed in temperature controlled rooms and given food and water ad libitum. Observations and measurements were made by an investigator blinded to the experimental group.

After measuring the baseline ear thickness, naïve mice were injected intraperitoneally (IP) with either 100 μl of PBS, or 100 μl of 3.50×10⁻⁵ M peptide solution (oxidized mTβ4 (“mTβ4-so”), or oxidized mLTβ4 (“mLTβ4-so”) [n=10 per group]). 30 minutes later, mice were injected intradermally with 40 μl of either PBS or 8.75×10⁻⁵ M solution of the appropriate peptide in both ears. One minute later, 40 nmol TPA (in 10 μl acetone) was applied to the anterior side of each ear. 6 h. and 24 h. after the topical application of TPA, the ears were re-measured, and the increases in ear thickness between baseline and 6 h. and between baseline and 24 h. calculated. For each mouse, increases in right and left ear thickness were averaged.

Statistics. Groups were compared for differences in the meaning using a one-tailed Student's t test; p<0.05 was considered statistically significant.

### Results

The methionated forms of Tβ4 were next compared for their ability to suppress ICD induced by topical application of TPA (12-O-tetradecanoylphorbol-13-acetate) to ear skin. We have previously shown that TPA-induced ICD is exaggerated in TCRδ^{-/-} mice [reference 4]. At 6 h. post-application of TPA, inflammation in mice treated with mUTβ4 was suppressed by 21% (p=0.0027) relative to PBS-treated controls (FIG. 3). By 24 hr, suppression was 13% (although this was not statistically significant). By contrast, there was a statistically significant suppression of inflammation by mLTβ4 at both of these time points: 41% (p<0.000001) at 6 h., and 24% (p=0.023) at 24 h., respectively. At 6 h., the suppression of ear swelling in mLTβ4-treated mice was significantly greater than that in mUTβ4-treated mice (18.00±1.58 vs. 24.10±1.83, p=0.011).

The results of this experiment are also summarized in Table 3.

TABLE 3
TPA Ear Thickness and P-Values
Methionated Thymosin Experiment

| | Thickness | | P values | |
|---|---|---|---|---|
| Time | (mm +/− SE) | | vs. PBS | vs. mTβ4-so |
| Six | PBS | 30.55 ± 0.92 | * | * |
| Hours | mTβ4 | 24.10 ± 1.83 | 2.72 × 10⁻³ | * |
| | mTβ4 | 18.00 ± 1.58 | 9.81 × 10⁻⁷ | 1.05 × 10⁻² |
| Twenty-Four | PBS | 21.95 ± 1.63 | * | * |
| Hours | mTβ4-so | 19.00 ± 1.83 | NS | * |
| | mTβ4-so | 16.75 ± 1.79 | 2.28 × 10⁻² | NS |

NS = not significant

### Example 5

### Activities of UTβ4 and LTβ4 in Allergic Contact Dermatitis (ACD)

Allergic Contact Dermatoses are dermatoses that arise from Type IV contact hypersensitivity reactions and include: poison ivy dermatitis, nickel dermatitis and chromate dermatitis (Janeway et al. (1994) Immunobiology: The Immune System in Health and Disease. Garland Publishing Inc: New York). In the ACD model used in this experiment, FVB wild-type mice are sensitized and challenged to DNFB, a hapten. Because of its simple chemical structure, DNFB can penetrate the skin with ease and bind to a protein carrier molecule. This hapten-protein complex is then processed by an antigen-presenting cell (APC), which displays pieces of antigen fragments on its MHC II receptor. From this point, immune T cells recognize and bind to the antigen-MHC II receptor and initiate a TH1-like cell mediated contact (Bellinghausen et. al. (1999) Signals involved in the early TH1/TH2 polarization of an immune response depending on the type of antigen. J Allergy Clin Immunol. 103:2 p. 298-306). This hypersensitivity starts 24 hours post exposure, peaks within 72 hours, and is characterized by a recruitment of T cells, phagocytes, and extravasation of fluid from vessels (Janeway, 1994, cited suppa).

### Materials and Methods

Experimental groups of 8-10 female FVB mice, 8-10 weeks of age, were housed in temperature controlled rooms and given food and water ad libitum. Observations and measurements were made by an investigator blinded to the experimental group.

Mice were sensitized on day 0 by epicutaneous application to razor-shaved abdominal skin of 25 μl of 0.5% DNFB in a mixture of acetone:olive oil (4:1). Before the challenge with DNFB, mice were injected (i) intraperitoneally with 100 μl of 3.50×10⁻⁵ M thymosin peptide solution (30 minutes before DNFB) and (ii) intradermally with 40 μl of 8.75×10⁻⁵ M peptide solution in both ears (1 min prior to DNFB challenge). On day 5, after measuring baseline ear thickness with an engineer's micrometer, mice were challenged by applying 10 μl of 0.2% DNFB in acetone:olive oil to each side of each ear. Ears were re-measured 6 h., 24 h., and 48 h. after challenge, and data expressed as the response above baseline (i.e., ear thickness 24 h. after challenge minus ear thickness immediately prior to challenge)±1 standard error of the mean (S.E.). For each mouse, increases in right and left ear thickness were averaged. The experiment was repeated twice.

Statistics. Groups were compared for differences in the meaning using a one-tailed Student's t test; p<0.05 was considered statistically significant.

### Results

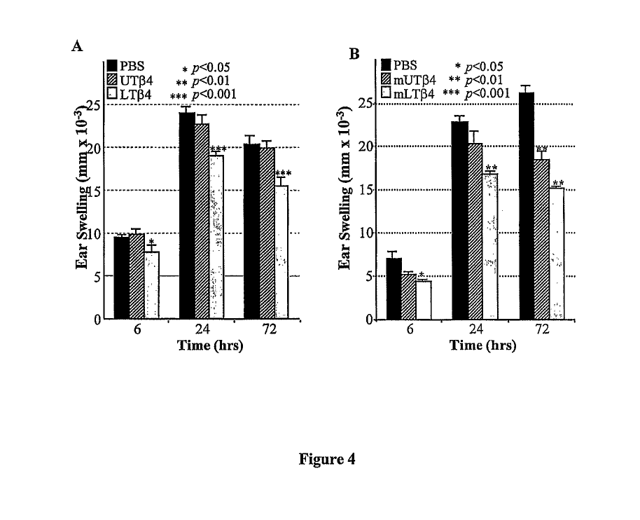

UTβ4 and LTβ4 were next compared to PBS for their ability to inhibit ACD inflammation induced by epicutaneous sensitisation of abdominal skin with of 25 μl of 0.5% DNFB followed by epicutaneous challenge to ear skein with 20 μl of 0.2% DNFB. As with ICD, ACD is highly exaggerated in TCRδ^{-/-} mice, and can be restored to normal levels by selective reconstitution with DETC [reference 4]. In no time point in this assay did UTβ4 show any significant anti-inflammatory activity: −4% (NS), 5% (NS), and 2% (NS) at 6 h., 24 h., and, 72 h, respectively (FIG. 4A). By contrast, LTβ4 suppressed the ACD ear swelling response by 17% (p=0.030), 21% (p=0.00031), and 24% (p=0.00052) at 6 h., 24 h., and, 72 h., respectively.

The effects were more pronounced when the methionated forms were compared for their ability to suppress ACD (FIG. 4B). Relative to PBS, mLTβ4 suppressed ear swelling by: 37% (p=0.042), 27% (p<0.0001), and 42% (p<0.00000001) at 6 h., 24 h., and 72 h., respectively, whereas mUTβ4 showed a significant effect only at 72 h. (29% lower than PBS, p=0.0040). The suppression of ACD observed with in LTβ4 was significantly greater than that observed with mUTβ4 at 24 h. and 72 h. (p=0.026 and 0.010, respectively). In summary, assessment of ACD-induced inflammation reveals that LTβ4 has significantly greater anti-inflammatory properties than does UTβ4, irrespective of whether or not the N-terminal methionine is retained.

At six hours, LTβ4-so treated mice had 21% thinner ears than uTβ4-so treated mice (7.83±0.61 vs. 9.85±0.66, p<0.05). At twenty-four hours, Lβ4-so treated mice had 16% thinner ears than uTβ4-so treated mice (18.98±0.74 vs. 22.68±1.33, p<0.05). At seventy-two hours, Lβ4-so treated mice had 22% thinner ears than uTβ4-so treated mice (15.45±0.91 vs. 19.90±1.02, p<0.005).

The results of this experiment are summarized in Table 4.

TABLE 4
DNFB Ear Thickness and P-Values

| Un-Methionated Thymosin Experiment | | | | | Methionated Thymosin Experiment | | | |
|---|---|---|---|---|---|---|---|---|
| | Thickness | | P values | | Thickness | | P values | |
| Time | (.01 mm +/− SE) | | vs. PBS | vs. uTβ4-so | (mm +/− SE) | | vs. PBS | vs. mTβ4-so |
| Six | PBS | 9.48 ± 0.55 | * | * | PBS | 7.00 ± 0.77 | * | * |
| Hours | uTβ4-so | 9.85 ± 0.66 | NS | * | mTβ4-so | 5.18 ± 0.78 | NS | * |
| | LTβ4-so | 7.83 ± 0.61 | 3.01 × 10⁻³ | 3.01 × 10⁻² | mTβ4-so | 4.39 ± 1.08 | 4.22 × 10⁻² | NS |
| Twenty- | PBS | 23.98 ± 0.95 | * | * | PBS | 22.79 ± 0.96 | * | * |
| Four | uTβ4-so | 22.68 ± 1.33 | NS | * | mTβ4-so | 20.25 ± 1.54 | NS | * |
| Hours | LTβ4-so | 18.98 ± 0.74 | 3.07 × 10⁻⁴ | 1.28 × 10⁻² | mTβ4-so | 16.75 ± 0.48 | 5.25 × 10⁻⁵ | 2.57 × 10⁻² |
| Forty- | PBS | 20.30 ± 1.25 | * | * | PBS | 26.08 ± 0.85 | * | * |
| Eight | uTβ4-so | 19.90 ± 1.02 | NS | * | mTβ4-so | 18.39 ± 1.67 | 3.97 × 10⁻³ | * |
| Hours | LTβ4-so | 15.45 ± 0.91 | 5.23 × 10⁻⁴ | 1.28 × 10⁻³ | mTβ4-so | 15.11 ± 0.37 | 3.85 × 10⁻⁸ | 1.02 × 10⁻² |

NS = not significant

### Example 6

### Endothelial Scratch Wound Assay

In order to determine whether LTβ4 can stimulate wound healing, we evaluated the effects of LTβ4 and uTβ4 on the in vitro migration of human umbilical vein endothelial cells (HUVECs) to close an artificial wound (scratch wound assay).

### Materials and Methods

The endothelial “scratch wound assay” was utilised to test the effects of mUTβ4 and mLTβ4 on the migration of endothelial cells in vitro, as described in Malinda et al. (1997) Thymosin beta 4 accelerates wound healing. J Invest Dermatol. 1999 September; 113(3):364-8 and Young et al., (1999) Thymosin b 4 sulfoxide is an anti-inflammatory agent generated by monocytes in the presence of glucocorticoids. Nature Medicine. 5:1424-1427. HUVECs were obtained from the Endothelial Cell Culture Core Laboratory of the Yale Skin Diseases Research Core Center (Dr. M. Kluger). Passage 3 was used, and plated at a density of 9.5×10⁴ cells per well. Cells were grown to 90% confluence in 24 well plates (Corning/Costar), in M-199 media supplemented with 20% FBS, antibiotics, and endothelial cell growth supplement (ECGS). A scratch was made along the diameter of each well with a sterile blue 1 ml pipette tip (Falcon). Cells were then rinsed in MEM buffer. Thymosin peptide was added to the cells in media lacking ECGS but containing 10 mM thymidine to inhibit cell proliferation. In the experimental groups, HUVECs were given 2.0×10-7 M peptide solution of mUTb4, or in LTb4. Control groups consisted of saline (negative control) and 200 mg of ECGS (positive control). The plates were incubated at 37° C., fixed in 2% paraformaldehyde, and stained with crystal violet at time points 9 h. and 24 h. The scratch width was measured using a microscope with a 4× objective lens connected to a digital camera and then analyzed using Scion Image 1.62 software. The percent closure of the 9 h. and 24 h. measurements was calculated by dividing by the original wound width at time zero in square pixels. For each group, the mean percent closure was calculated, based on 4 images per well, 4 wells per group.

### Results

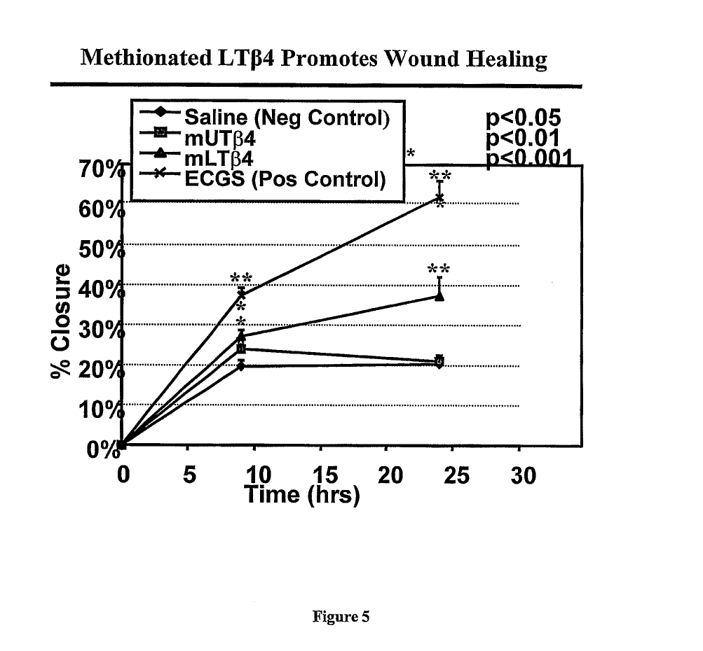

In the Scratch Wound experiment, compared to the PBS control's promotion of endothelial migration of 19.7% mLβ promoted migration by: (27.2%, p<0.05), and (37.3%, p<0.05) while mTβ4 promoted migration by: (24%, p<0.05) and (21%, NS) at nine, and twenty-four hours respectively. At twenty four hours, mLβ4 promoted 15.3% more closure than mTβ4-so treated mice (37.3±.4.4 vs. 21.0±1.5, p<0.01). HUVECs treated with 200 μg of ECGS showed significantly more closure (37.4%, p<0.01; 61.9% p<0.01) than all other group s at nine and twenty-four respectively. The results of this experiment are shown in FIG. 5.

The results of this experiment are also summarized in Table 5.

TABLE 5
Wound Closure and P-Values
Scratch Wound Assay: Methionated Thymosin Experiment

| | % Closure | | P values | | |
|---|---|---|---|---|---|
| Time | (% +/− SE %) | | vs. PBS | vs. mTβ4 | vs. mLTβ4 |
| Nine | PBS | 19.7 ± 1.5 | * | * | * |
| Hours | mTβ4 | 24.0 ± 1.1 | 3.89 × 10⁻² | * | * |
| | mLTβ4 | 27.2 ± 1.6 | 1.23 × 10⁻² | NS | * |
| | ECGS | 37.4 ± 1.7 | 6.78 × 10⁻⁴ | 1.36 × 10⁻³ | 5.89 × 10⁻³ |
| Twenty- | PBS | 20.3 ± 0.8 | * | * | * |
| Four | mTβ4 | 21.0 ± 1.5 | NS | * | * |
| Hours | mLTβ4 | 37.3 ± 4.4 | 4.54 × 10⁻³ | 5.77 × 10⁻³ | * |
| | ECGS | 61.9 ± 3.8 | 1.91 × 10⁻⁵ | 2.61 × 10⁻⁵ | 2.69 × 10⁻³ |

NS = not significant

Discussion of Results Presented in Examples

The data presented in the example, confirm that oxidised UTβ4 has anti-inflammatory properties as well as stimulatory effects on endothelial cell migration. However, the anti-inflammatory activities of UTβ4 were selective for particular assays as evidenced by the lack of activity in response to ACD. By contrast, oxidised LTβ4 demonstrated significant anti-inflammatory activity in all three assays employed. Thus, LTβ4 has the potential to be a potent immunological effector produced by lymphocytes. The hitherto unrecognised and potent activity of LTβ4 in ACD is particularly provocative since this is a common, clinically-relevant condition, primarily regulated by the lymphoid compartment.

In their original studies, Young and colleagues showed a dose-dependent anti-inflammatory effect of oxidized UTβ4 following λ-carrageenan injection into mouse footpads [reference 8]. It was suggested that the anti-inflammatory activity of oxidized UTβ4 (“UTβ4-so”) serves as a safety-feedback signal, its oxidation reflecting an oxidizing environment that often correlates with host cell damage. Since oxidative damage may be reversed by methionine sulfoxide reductase, the oxidation and reduction of the methionine residue on UTβ4 might well act as a sensitive regulatory sentinel [reference 8]. Indeed, the feedback mechanism provoked by oxidation of methionine residue six (M6) in UTβ4 may itself promote methionine sulfoxide reductase and the repair of oxidative damage. In this regard, LTβ4, with an additional oxidisable methionine residue (Table 1), has a potentially greater capacity to reduce oxidative stress. Such a mechanism may account for the greater anti-inflammatory activity seen in our comparative analyses of LTβ4 versus UTβ4.

Those skilled in the art will recognize, or be able to ascertain using no more than routine experimentation, numerous equivalents to the specific method and reagents described herein, including alternatives, variants, additions, deletions, modifications and substitutions. Such equivalents are considered to be within the scope of this invention and are covered by the following claims

### REFERENCES

1. Hayday A C. γδ cells: a right time and a right place for a conserved third way of protection. Ann Rev Immunol 2000; 18:975-1026.
2. Girardi M, Oppenheim D, Lewis J, Filler R, Tigelaar R E, and Hayday A C. The regulation of squamous cell carcinoma development by γδ T cells Science 2001; 294:605-609.
3. Jameson J, Ugarte K, Chen N, Yachi P, Fuchs E, Boismenu R, Havran W L. A role for skin gammadelta T cells in wound repair. Science 2002; 296:747-9.
4. Girardi M, Lewis J, Glusac E, Filler R B, Geng L, Hayday A C, Tigelaar R E. Resident skin-specific gamma delta T cells provide local, non-redundant regulation of cutaneous inflammation. J Exp Med 2002; 195: 855-67.
5. Shiohara T, Moriya N, Hayalcawa J, Itohara S, Ishikawa H. Resistance to cutaneous graft-vs.-host disease is not induced in T cell receptor δ gene-mutant mice. J Exp Med 1996; 183:1483-1489.
6. Shires J, Theodoridis E, Hayday A C. Biological insights into murine TCR γδ⁽⁺⁾ and TCR αβ⁽⁺⁾ intraepithelial lymphocytes provided by the serial analysis of gene expression. Immunity 2001; 15:419-434.
7. Huff T, Muller C S, Otto A M, Netzker R, Hannappel E. β Thymosins, small acidic peptides with multiple functions. Int J Biochem Cell Biol 2001; 33:205-20.
8. Young J D, Lawrence A J, MacLean A G, Leung B P, McInnes I B, Canas B, Pappin D J C, Stevenson R D. Thymosin β4 sulfoxide is an anti-inflammatory agent generated by monocytes in the presence of glucocorticoids. Nature Medicine 1999; 5:1424-1427.
9. Malinda K M, Goldstein A L, Kleinman H K. 1997. Thymosin beta 4 stimulates directional migration of human umbilical vein endothelial cells. FASEB J 1997; 11:474-81.
10. Malinda K M, Sidhu G S, Mani H, Banaudlia K, Maheslhwari R K, Goldstein A L, Kleinman H K. Thymosin beta 4 accelerates wound healing. J Invest Dermatol 1999; 113:364-8.
11. Gomez-Marquez J, Dosil M, Segade F, Bustelo X R, Pichel J G, Dominguez F, Freire M. 1989 Thymosin-beta 4 gene. Preliminary characterization and expression in tissues, thymic cells, and lymphocytes. J. Immunol. 143:2740-4.
12. Rudin C M, Engler P, Storb U. Differential splicing of thymosin beta 4 mRNA. J Immunol 1990; 144: 4857-62.
13. Li X, Zimmerman A, Copeland N G, Gilbert D J, Jenkins N A, Yin H L. The mouse thymosin beta 4 gene: structure, promoter identification, and chromosome localization. Genomics 1996; 32:388-94.
14. Nixon-Fulton J L, Bergstresser P R, Tigelaar R E. Thy-1⁺ epidermal cells proliferate in response to concanavalin A and interleukin 2. J Immunol 1986; 136:2776-86.
15. Black D L. Protein diversity from alternative splicing: a challenge for bioinformatics and post-genome biology. Cell 2000, 103:367-70.
16. Roberts S J, Smith A L, West A B, Wen L, Findly R C, Owen M J, Hayday A C. T-cell αβ⁺ and γδ⁺ deficient mice display abnormal but distinct phenotypes toward a natural, widespread infection of the intestinal epithelium. Proc Natl Acad Sci USA 1996; 93:11774-9.
17. Poussier P, Ning T, Banerjee D, Julius M. A unique subset of self-specific intraintestinal T cells maintains gut integrity. J Exp Med 2002; 195:1491-7.
18. Pazmino N H, Ihle, J N, McEwan, R N, Goldstein, A L. Control of differentiation of thymocyte precursors in the bone marrow by thymic hormones. Cancer Treat. Rep 1978; 62: 1749.
19. Thurman, G B, Low TLK, Rossio, J L, Goldstein, A L. Specific and nonspecific macrophage migration inhibition. In: Goldstein A L, Chirigos M, eds. Lymphokines and thymic factors. Their potential in cancer therapeutics. New York: Raven Press, 1981:145.
20. Weber A, Nachmias V T, Pennise C R, Pring M, Safer D. Interaction of thymosin beta 4 with muscle and platelet actin: implications for actin sequestration in resting platelets. Biochemistry 1992; 31: 6179-6185.
21. De La Cruz E M, Pollard T D. Structural biology: Actin' up. Science 2001; 293:616-8.
22. Vancompernolle K, Goethals M, Huet C, Louvard D, Vandelcerclclove J. G- to F-actin modulation by a single amino acid substitution in the actin binding site of actobindin and thyinosin beta 4. EMBO J 1992; 11: 4739-46.
23. Derossi D., Joliot A H, Chassaing G, Prochiantz A. The third helix of the antennapedia homeodoinain translocates through biological membranes. J. Biol Chem 1994; 269:10444-50.
24. Vives E, Brodin P, Lebleu B. A truncated HIV-1 Tat protein basic domain rapidly translocates through the plasma membrane and accumulates in the cell nucleus. J Biol Chem 1997; 272:16010-7.

1. A method of treating an inflammatory condition in a subject comprising administering to the subject a therapeutically effective amount of a composition comprising a lymphoid thymosin-β4 polypeptide.

2. The method of claim 1, wherein the inflammatory condition is inflammatory bowel disease, joint disease, lung disease, skin condition, or vasculitis.

3.-15. (canceled)

16. The method of claim 1, wherein the lymphoid thymosin-β4 polypeptide comprises the amino acid sequence of SEQ ID NO:8 or SEQ ID NO:9.

17. The method of claim 1, wherein the lymphoid thymosin-β4 is linked to a protein transduction sequence.

18.-20. (canceled)

21. The method of claim 1, wherein the lymphoid thymosin-β4 polypeptide is isolated from lymphoid tissue, leukocytes or lymphocytes, or from cells or tissue of the lympho-endoreticular system.

22.-24. (canceled)

25. The method of claim 1, wherein the lymphoid thymosin-β4 polypeptide is a synthetic polypeptide.

26. The method of claim 1, wherein the lymphoid thymosin-β4 polypeptide is oxidized.

27. (canceled)

28. The method of claim 1, wherein the composition is administered topically.

29. The method of claim 28, wherein the composition is in the form of a solution, gel, creme, paste, lotion, spray, suspension, dispersion, salve, hydrogel, a liposome, or ointment formulation.

30. (canceled)

31. The method of claim 1, further comprising administering an anti-inflammatory compound, a corticosteroid, a retinoid an antibiotic or a cyclosporine derivative.

32.-33. (canceled)

34. A method of promoting wound healing in a subject comprising administering to the subject a therapeutically effective amount of a composition comprising a lymphoid thymosin-β4 polypeptide.

35. The method of claim 34, wherein the lymphoid thymosin-β4 polypeptide comprises the amino acid sequence of SEQ ID NO:8 or SEQ ID NO:9.

36. The method of claim 34, wherein the lymphoid thymosin-β4 is linked to a protein transduction sequence.

37.-39. (canceled)

40. The method of claim 34, wherein the lymphoid thymosin-β4 polypeptide is isolated from lymphoid tissue, from leukocytes or lymphocytes, or from cells or tissue from the lympho-endoreticular system.

41.-43. (canceled)

44. The method of claim 34, wherein the lymphoid thymosin-β4 polypeptide is a synthetic polypeptide.

45. The method of claim 34, wherein the lymphoid thymosin-β4 polypeptide is oxidized.

46. (canceled)

47. The method of claim 34, wherein the composition is administered topically.

48. The method of claim 47, wherein the composition is in the form of a solution, gel, creme, paste, lotion, spray, suspension, dispersion, salve, hydrogel, a liposome, or ointment formulation.

49. (canceled)

50. The method of claim 34, further comprising administering an anti-inflammatory compound, a corticosteroid, a retinoid, an antibiotic or a cyclosporine derivative.

51.-62. (canceled)

63. A method of treating an inflammatory condition in a subject comprising administering to the subject a therapeutically effective amount of a nucleic acid encoding a lymphoid thymosin-β4 polypeptide.

64.-65. (canceled)


SEQUENCE LISTING (9 sequences)

SEQ ID NO: 1 - protein, 43 aa, Homo sapiens
SDKPDMAEIEKFDKSKLKKTETQEKNPLPSKETIEQEKQAGES

SEQ ID NO: 2 - protein, 44 aa, Homo sapiens
MSDKPDMAEIEKFDKSKLKKTETQEKNPLPSKETIEQEKQAGES

SEQ ID NO: 3 - DNA, 26 bp, Artificial Sequence
  actin forward primer
tccctgtatgcctctggtcgtaccac

SEQ ID NO: 4 - DNA, 27 bp, Artificial Sequence
  actin reverse primer
caggatcttcatgaggtagtctgtcag

SEQ ID NO: 5 - DNA, 23 bp, Artificial Sequence
  LT 4 forward primer
tgcctgtccagcgcaggcacttg

SEQ ID NO: 6 - DNA, 23 bp, Artificial Sequence
  UT 4 forward primer
cttctgagcagatcagactctcc

SEQ ID NO: 7 - DNA, 24 bp, Artificial Sequence
  T 4 (common) reverse primer
ctctgctagccagaccatcagatg

SEQ ID NO: 8 - protein, 49 aa, Homo sapiens
LLPATMSDKPDMAEIEKFDKSKLKKTETQEKNPLPSKETIEQEKQAGES

SEQ ID NO: 9 - protein, 50 aa, Homo sapiens
MLLPATMSDKPDMAEIEKFDKSKLKKTETQEKNPLPSKETIEQEKQAGES